(19) United States
(12) Patent Application Publication
(10) Pub. No.: US 2026/0334977 A1
(43) Pub. Date: Oct. 29, 2026

(54) MOTORIZED STRUT WIRELESS COMMUNICATION

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Brian Roberts, Germantown, TN (US); Sied W. Janna, Memphis, TN (US); Paul Bell, Vestavia Hills, AL (US); Michael-Julien Santini, Millington, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/117,693

(22) PCT Filed: Nov. 8, 2023
(86) PCT No.: PCT/US2023/037005

§ 371 Date: Apr. 2, 2025

Related U.S. Application Data
(60) Provisional application No. 63/424,224, filed on Nov. 10, 2022.

(51) Int. Cl. A61B 17/62; A61B 17/00; A61B 34/10; H04W 4/80; H04W 76/14

(52) U.S. Cl. CPC A61B 17/62; A61B 34/10; H04W 4/80; H04W 76/14; A61B 2017/00221

(57) ABSTRACT

A spatial frame apparatus for positioning on a patient to treat an injury. The apparatus includes a frame having one or more motor modules including one or more motorized struts. One or more motorized struts include one or more motors configured to cause movement of one or more motorized struts in one or more directions in accordance with one or more treatment plans. At least one motor module is wirelessly coupled to at least another motor module. The motor modules include one or more processing components communicatively coupled to one or more motorized struts and/or one or more motors. The processing components execute at least one adjustment of one or more motorized struts based on the treatment plans and in accordance with one or more wireless signals received from one or more motor modules and/or one or more external devices communicatively coupled to one or more motor modules.

### CROSS-REFERENCE TO RELATED APPLICATIONS

This is a non-provisional of, and claims the benefit of the filing date of, U.S. provisional patent application number 63/424,224, filed Nov. 10, 2022, entitled “Motorized Strut Wireless Communication,” the entirety of which is incorporated by reference herein.

### TECHNICAL FIELD

The present disclosure relates generally to orthopedic devices, systems, and methods for facilitating fracture alignment such as the treatment of musculoskeletal conditions with a spatial frame, which includes a motorized strut such as, for example, a geared-motor assembly, configured to include wireless communication capabilities among its various components and/or external devices.

### BACKGROUND

People suffer bone fractures each year. In many instances, a person that suffers a bone fracture is required to use a bone alignment device such as, for example, an external fixation system, a spatial frame, a hexapod, etc. (terms used interchangeably herein without the intent to limit or distinguish) to align two or more bones, bone fragments, bone pieces, etc. (terms used interchangeably herein without the intent to limit or distinguish). Generally speaking, spatial frames allow for polyaxial movement of the coupled bones and are typically used to keep fractured bones stabilized and in alignment during a treatment period.

Generally speaking, the spatial frame includes first and second rings, platforms, frames, bases, etc. (terms used interchangeably herein without the intent to limit or distinguish) intercoupled by a plurality of struts. In use, the struts have adjustable lengths that may be manually adjusted regularly (e.g., daily) in accordance with a prescription or treatment plan (terms used interchangeably herein without the intent to limit or distinguish). As the lengths of the struts are adjusted, the platforms may be brought closer together or moved farther apart. The treatment plan specifies strut length adjustments to be made over time to ensure successful bone alignment. However, some existing systems are not capable of providing wireless communication capabilities among its components and/or with external devices.

### SUMMARY

This summary is provided to introduce a selection of concepts in a simplified form that are further described below in the detailed description. This summary is not intended to identify key features or essential features of the claimed subject matter, nor is it intended as an aid in determining the scope of the claimed subject matter.

In some examples, the present disclosure relates to a spatial frame apparatus for positioning on a patient to treat an injury. The apparatus may include a frame having one or more motor modules including one or more motorized struts. One or more motorized struts may include one or more motors configured to cause movement of one or more motorized struts in one or more directions in accordance with one or more treatment plans. At least one motor module may be configured to be wirelessly coupled to at least another motor module. The motor modules may also include one or more processing components communicatively coupled to one or more motorized struts and/or one or more motors. The processing components may be configured to execute at least one adjustment of one or more motorized struts based on the treatment plans and in accordance with one or more wireless signals received from one or more motor modules and/or one or more external devices communicatively coupled to one or more motor modules.

In any preceding or subsequent examples, the present disclosure may be configured to include one or more of the following optional features. Each motor module may include one or more wireless communication components configured to wirelessly communicate with one another and/or with one or more external devices.

In any preceding or subsequent examples, the wireless communication components may be configured to be paired with one another and/or with one or more external devices.

In any preceding or subsequent examples, the wireless communication components may be configured to wirelessly communicate with one another using one or more communication protocols. The communication protocols may include at least one of the following: Bluetooth™ communication protocol, Bluetooth™ Low Energy (BLE) communication protocol, near field communication (NFC) protocol, and any combination thereof.

In any preceding or subsequent examples, at least one motor module may be selected as a primary motor module and other motor modules may be selected as secondary motor modules. The primary motor module may be configured to transmit instructions to each and/or one or more of the secondary motor modules for execution of the treatment plan.

In any preceding or subsequent examples, one or more external devices may be configured to be wirelessly coupled to the primary motor module, where the primary motor module is configured to receive instructions from one or more external devices and transmit received instructions to one or more secondary motor modules.

In any preceding or subsequent examples, one or more external devices may include a software application for at least one of: monitoring, controlling, and/or adjusting operation of one or more motor modules using wireless communications.

In any preceding or subsequent examples, one or more motors may be configured to cause one or more motorized struts to execute an adjustment in accordance with the treatment plan. The adjustment may include at least one of the following: a direction of adjustment of one or more motorized struts, a length of adjustment of one or more motorized struts, an angle of adjustment of one or more motorized struts, a time for adjustment of one or more motorized struts and/or any other parameters for changing positioning of one or more motorized struts, and/or any combination thereof.

In any preceding or subsequent examples, one or more motor modules may be configured to receive one or more firmware updates using the one or more wireless signals. One or more processing components may be configured to authenticate a source of one or more firmware updates, verify firmware updates, and install, upon authentication and verification of the firmware updates, the firmware updates on an operating computing system of one or more motor modules. Further, the processing components may perform at least one of: authentication of the source of the firmware update(s), verification of the firmware update(s), and installation of the firmware update(s) without interruption of execution of at least one adjustment of one or more motorized struts based on one or more treatment plans.

Examples of the present disclosure provide numerous advantages. For example, by providing an ability to wireless couple motor modules associated with each of the motorized struts, the present disclosure may be configured to obviate a need for connecting motorized modules using wires. Avoiding wired connections on the spatial frame may be configured to reduce an amount of space that is used by the frame, wires, and/or its other components. It may also decrease overall weight of the spatial frame. Further, lack of wires may prevent wire-snagging on objects around the patient. Moreover, wireless transmission of data (including the treatment plan, monitoring data, loading data, etc.) to and from the spatial frame and between its components may be advantageous in that the patient might not have to come to a doctor's office for adjustments to the frame's motorized struts, as an external device may be used to monitor the treatment, perform adjustments and/or control operation of the motorized frame.

Further features and advantages of at least some of the examples of the current subject matter, as well as the structure and operation of various examples of the current subject matter, are described in detail below with reference to the accompanying drawings.

### BRIEF DESCRIPTION OF THE DRAWINGS

The accompanying drawings, which are incorporated in and constitute a part of this specification, show certain features of the subject matter disclosed herein and, together with the description, help explain some of the principles associated with the disclosed implementations. In the drawings, FIG. 1 illustrates a perspective view of a conventional spatial frame including first and second platforms and a plurality of manually adjustable struts coupled thereto;

FIG. 2 illustrates a cross-sectional view of an example of a motorized strut that may be used in a spatial frame such as, for example, within the spatial frame shown in FIG. 1;

FIG. 3A illustrates a perspective view of an alternate conventional spatial frame including first and second platforms and a plurality of manually adjustable struts coupled thereto;

FIG. 3B illustrates a perspective view of an example of a spatial frame including a plurality of geared-motor assemblies coupled to the manually adjustable struts of the spatial frame in FIG. 3A in accordance with one or more features of the present disclosure, the spatial frame including the plurality of geared-motor assemblies and a companion APP to transmit and receive data, instructions, and updates;

FIG. 4 illustrates a side view of a conventional manually adjustable strut;

FIGS. 5A and 5B illustrate various views of an example of a geared-motor assembly in accordance with one or more features of the present disclosure, the geared motor assembly being coupled to a manually adjustable strut;

FIG. 6 illustrates an exemplary control circuit of a spatial frame;

FIG. 7 is a block diagram of a system that may be used with a spatial frame (as, for example, is shown in FIGS. 1-6), according to some examples of the present disclosure;

FIG. 8 illustrates a process for executing wireless communications using a spatial frame, according to some examples of the present disclosure;

FIG. 9 illustrates a process for executing updates to firmware of a spatial frame, according to some examples of the present disclosure;

FIG. 10 illustrates an exemplary computing apparatus, according to some examples of the present disclosure;

FIG. 11 illustrates an example of a storage medium to store spatial frame logic, according to some examples of the present disclosure; and

FIG. 12 illustrates an example computing platform, according to some examples of the present disclosure.

It should be understood that the drawings are not necessarily to scale and that the disclosed examples are sometimes illustrated diagrammatically and/or in partial views. In certain instances, details that are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

To address these and potentially other deficiencies of currently available solutions, one or more implementations of the present disclosure relate to methods, systems, articles of manufacture, and the like that can, among other possible advantages, provide a spatial frame having a geared-motor assembly and further configured to provide wireless communication capabilities to one or more of its components and/or with one or more external devices.

### I. Spatial Frame

Spatial frames are well known. One known example of a spatial frame is the TAYLOR SPATIAL FRAME® manufactured and sold by Smith Nephew, Inc. As illustrated in FIG. 1 an example spatial frame 100 may form a hexapod having a circular, metal frame with a first platform 102 and a second platform 104 connected by six adjustable length struts 106 (labeled as struts 106-1 through 106-6 in FIG. 1). Each strut 106 may be independently lengthened or shortened relative to the rest of the frame, thereby allowing for six different axes of movement.

Each strut 106 may include an outer body and an inner body, which may be configured as, or be operatively coupled to, a threaded rod (also referred to as a lead screw). The outer body may be coupled to one of the platforms, such as, the second platform 104 by way of a joint as shown. The inner body may be coupled to the other platform, such as, the first platform 102 by way of a joint as shown. To lengthen or shorten one of struts 106, the outer body and the inner body may be moved or translated relative to one another. For example, in some examples, the strut 106 may include an adjustment nut wherein rotation of the adjustment nut moves the inner body (e.g., threaded rod or lead screw) relative to the outer body to adjust an overall length of the strut.

In use, the spatial frame 100 may be used to treat a variety of skeletal fractures of a patient. Typically, the spatial frame 100 is positioned around the patient and is used to align two or more bone portions. To do so, a length of each strut 106 may be incrementally adjusted (e.g., shortened or lengthened) in accordance with a treatment plan that specifies adjustments to be made to each strut 106 over time to ensure successful bone alignment. In many instances, the length of each strut 106 should be adjusted daily to comply with the provided treatment plan. Adjusting the length of each strut 106 adjusts the distance between the first and second platforms 102, 104, and hence the first and second bone portions coupled thereto.

During use, patient's bones are normally adjusted (e.g., lengthened, shortened, etc.) by adjusting struts manually, for example, by hand or using a wrench to move the bone segments at a rate of approximately 1 mm/day, which is then proceeded by a consolidation phase before the spatial frame is removed.

It is theoretically known to automate and/or motorize adjustment of a spatial frame by motorizing or otherwise automating strut adjustments. For example, one known motorized strut is the Robotic Hexapod System manufactured by Orthospin Ltd. In use, the Robotic Hexapod System includes an offset motor design that engages custom struts positioned between the first and second platforms. That is, the motor includes a spur gear engaged with a second spur gear associated with the threaded rod of the strut. In use, rotation of the motor drives rotation of the threaded rod via the interaction between the spur gears. The Robotic Hexapod System however suffers from a number of disadvantages including being very bulky and having trailing cables.

However, currently commercially available spatial frames are dependent on manual adjustment of each strut. As a result of the requirement for manual adjustments, generally speaking, successful treatment requires patient compliance (e.g., daily manual adjustments to each of the struts) to avoid human error. In routine clinical practice, the treatment plan may require multiple daily adjustments to be made to each of the plurality of struts. For example, a patient may be required to manually adjust one or more of the struts, typically two or more times each day, and often over long periods of time with support from either a family member, a clinician, or both. As such, compliance with the treatment plan may be burdensome, painful, etc.

As a result, the number of adjustments dictated by the treatment plan may be limited. For example, generally speaking, treatment plans often limit the required number of daily adjustments to each of the plurality of struts to four per day. During a normal treatment plan, this may equate to approximately 720 adjustments (e.g., turns) over a 1-month treatment span (e.g., 6 struts×4 adjustments per day×30 days). During an extended treatment plan for more severe applications, this may equate to approximately 2,160 adjustments (e.g., turns) over a 3-month treatment span (e.g., 6 struts×4 adjustments per day×90 days).

In addition, during the treatment period, the patient may require numerous clinical visits to confirm proper strut adjustments to ensure compliance and avoid incorrect adjustment, which has historically been the leading cause of treatment failure.

Motorized and/or automated struts could provide numerous advantages over manually adjustable struts. In use, electric motors, motor-drive units, and a control unit (e.g., a central control unit) could function to supersede the manual actuation of the strut adjustments. For example, a motorized system could eliminate the need for patient compliance and decrease the frequency of post-operative visits for patient supervision given that the spatial frame only has to be activated at the start of the distraction phase and terminated at the end of the distraction phase without any patient intervention. Additionally, automatic distraction could enable a higher distraction frequency and result in smaller excursions per activation. Smaller distraction steps or adjustments have the potential to result in less damage to the distracted tissues, improving bone regeneration and adaptation of the surrounding soft tissues. That is, spatial frames equipped with motorized and/or automated struts offer the potential to increase the number of daily distraction adjustments by enabling finer (e.g., smaller) adjustments at a controllable rate and frequency of distraction that encourages better quality bone formation. Making finer (e.g., smaller) adjustments during limb lengthening can have significant advantages in terms of reduced soft tissue damage, less pain, and opioid usage and accelerated bone healing. One study has found that the bone fixation index was only 5-6 days/cm when using motorized and/or automated distraction compared to 22-24 days/cm by manual adjustment.

In some examples, a motorized strut could be programmed to perform anywhere from 1 adjustment per day to continuous adjustments. In some examples, finer adjustments can increase the number of adjustments over a 1-month period from approximately 720 adjustments to approximately 3,600 adjustments (e.g., 6 struts×20 adjustments per day×30 days). In another example, finer adjustments can increase the number of adjustments over a 1-month period to approximately 259,200 adjustments (e.g., 6 struts×1440 adjustments per day×30 days). Over an extended 3-month treatment period, this could increase the number of adjustments from approximately 2,160 adjustments to approximately 10,800 adjustments (e.g., 6 struts×20 adjustments per day×90 days). In another example, finer adjustments can increase the number of adjustments over a 3-month period to approximately 777,600 adjustments (e.g., 6 struts×1440 adjustments per day×90 days).

In use, each motorized strut may include a motor and may be used in a spatial frame such as, for example, spatial frame 100, to move the first and second platforms 102, 104, respectively, to align two or more bone portions. In use, the spatial frame and/or system architecture may be arranged and configured to automatically adjust the motorized struts according to the prescribed treatment plan (e.g., automatically adjust the plurality of motorized struts without patient intervention). Alternatively, the spatial frame and/or system architecture may be arranged and configured to require patient and/or caregiver activation to begin the process of automatically adjusting the motorized struts according to the prescribed treatment plan. For example, the spatial frame may be arranged to intermittently auto-adjust the motorized struts at predetermined times according to the treatment plan. Alternatively, the spatial frame may be arranged to intermittently auto-adjust the motorized struts at select times when convenient and/or selected by the patient. Alternatively, the spatial frame may be arranged and configured to continuously auto-adjust the motorized struts in small discrete increments.

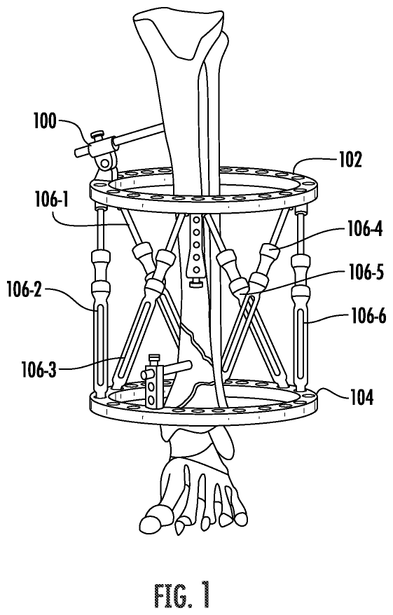

Referring to FIG. 2, an example of a motorized strut 200 is shown. In use, the motorized strut 200 may be coupled to first and second platforms in a spatial frame. For example, the motorized strut 200 may be used in place of the manually adjustable struts 106 shown in FIG. 1. As shown in FIG. 2, the motorized strut 200 may include an outer body 202 operatively coupled with a first joint 204 for coupling to a first platform, an inner body 210 operatively coupled with a second joint 212 for coupling to a second platform, and a drive mechanism, actuator, etc. 220 (used interchangeably herein without the intent to limit or distinguish). In use, actuation of the drive mechanism 220 moves the inner body 210 relative to the outer body 202 to adjust a length of the motorized strut 200.

As illustrated, the drive mechanism 220 may include a motor 222 and a threaded rod or lead screw 224 arranged and configured so that, in use, actuation of the motor 222 rotates the threaded rod 224, which moves the inner body 210 relative to the outer body 202 to adjust an overall length of the motorized strut 200. In addition, the drive mechanism 220 may include one or more gears to adjust speed and torque of the motor 222.

In addition, the motorized strut 200 may include any required circuity. For example, the motorized strut 200 may include one or more position sensors to, for example, monitor absolute position or length of the motorized strut 200. In addition, and/or alternatively, the motorized strut 200 may include other sensors for monitoring various biomechanical parameters such as, for example, a force sensor 230 for monitoring stresses and forces, across the bone gap and/or the soft tissues (muscle, apposing cartilage or peripheral sensory nerves), an accelerometer for capturing patient ambulation data (steps, distance, speed and cadence), a gyroscope for measuring the degree of alignment between the bone fragments, and a sensor motor support 232, etc. In addition, and/or alternatively, the motorized strut 200 may include an encoder such as, for example, a rotary encoder for measuring rotation of the motor 222 for accurate positioning and motion control. In addition, and/or alternatively, the motorized strut 200 may include flash memory for storing unique identifiers (e.g., addresses) and for storing current position, biomechanical and ambulatory data, etc.

As illustrated, the motorized strut 200 may be arranged and configured with an in-line design, wherein the motor 222 shares a common longitudinal axis as the threaded rod 224 and the telescoping portion (e.g., inner body 210) (e.g., the motor and electronics are housed in an enclosure or body that shares the same axis as the threaded rod, adjustment nut, and telescoping portion of the strut).

Additional information on examples of motorized spatial frames can be found in International Patent Application No. PCT/US20/52276, filed on Sep. 23, 2020, entitled “Automated Spatial Frame and Automated Struts Used Therewith,” the entire contents of said application being hereby incorporated by reference in its entirety herein.

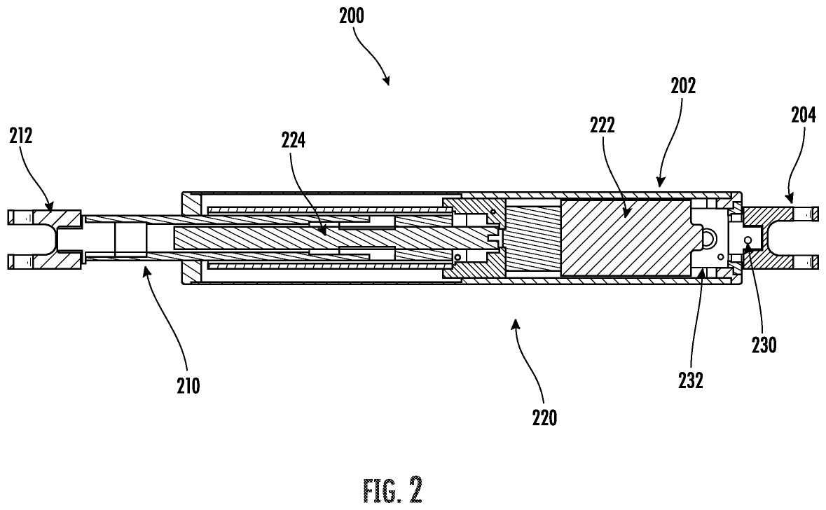

With reference to FIGS. 3A-5B, in some examples, the motorized strut may be a geared-motor assembly. The geared-motor assembly may be arranged and configured as a self-contained unit arranged and configured to receive and transmit data with an external computing system. The geared-motor assembly may include an enclosure or housing containing a motor, a power supply, a microprocessor, and all other power and control circuity needed to engage and control a manually adjustable strut in a spatial frame.

In some examples, each geared-motor assembly may include a motor and a torque transmitting mechanism such as, for example, a gear, arranged and configured to engage a corresponding gear on a manually adjustable strut in a spatial frame. In use, actuation of the motor enables motorized rotation of the torque transmitting mechanism and thus the manually-adjustable strut coupled to the geared-motor assembly. In addition, each geared-motor assembly may include a microcontroller arranged and configured to control operation of the geared-motor assembly including, for example, receiving and/or updating a treatment plan and/or controlling activation of the motor without the need for a separate centralized master control unit positioned within the spatial frame. Each geared-motor assembly may further include a wireless communication chip or antenna arranged and configured to communicate with an external computing system to, for example, exchange data relating to strut position, exchange data relating to and updating the prescribed treatment plan, etc. Each geared-motor assembly may also include a power supply such as, for example, batteries, to power the geared-motor assembly including, for example, the motor, the microcontroller, the wireless communication chip, and any associated sensors and/or additional circuity. Each geared-motor assembly may also include a sensor for either positional control, biomechanical feedback, or a fault level detection in the gear train. In some examples, each geared-motor assembly may include its own self-contained power management, wireless communication, and microcontroller unit that controls the position of the strut.

In some examples, the geared-motor assemblies are arranged and configured to be used in a spatial frame. The spatial frame includes a plurality of manually adjustable struts coupled to first and second platforms. Movement of the struts move the first and second platforms, and hence the first and second bone portions coupled thereto. FIG. 3A illustrates an example of a spatial frame 300. The spatial frame 300 includes a first platform such as, for example, first platform 102, a second platform such as, for example, second platform 104, and a plurality of manually adjustable struts such as, for example, struts 106, coupled to the first and second platforms 102, 104.

Each of the geared-motor assemblies may be coupled to one of the manually adjustable struts. Thus arranged, the spatial frame can be selectively configured to operate in either of a first or manually adjustable mode or configuration of operation wherein each strut may be manually adjusted or a second or motorized mode or configuration of operation wherein a geared-motor assembly may be coupled to each strut to facilitate motorized adjustment of the struts. In addition, by utilizing a geared-motor assembly that couples to a manually adjustable strut by, for example, interconnecting corresponding gears, an offset motor design is achieved thereby enabling a shorter minimum strut length to be achieved (e.g., the geared-motor assemblies may be arranged and configured with a shorter minimum length (e.g., length of the strut as measured end to end (e.g., joint to joint) with the threaded rod assembly in the fully retracted position)) as compared to conventional in-line motorized struts, while still providing a reasonable working length (e.g., adjustment length of the strut in use-length adjustment or difference between the minimum length and the maximum length of the strut).

As shown in FIG. 3B, during use, the removable geared-motor assembly 302 is arranged and configured to engage, attach, couple, etc. to the manually adjustable struts 106 of the spatial frame 300. Thus arranged, the spatial frame 300 can be operated in and switched between two modes or configurations of operation. In the first mode or configuration of operation, the struts 106 may be manually adjustable, as illustrated in FIG. 3A. In the second mode or configuration of operation, a geared-motor assembly 302 may be attached to one or more of the manually adjustable struts 106 to enable motorized and/or automated adjustment of the struts.

The geared-motor assemblies 302 are coupled to the manually adjustable struts 106 of the spatial frame 300. In some examples, the geared-motor assemblies 302 may be coupled to the manually adjustable struts 106 after surgery in clinic by, for example, a primary care provider. Alternatively, or in addition to, the geared-motor assemblies 302 may be coupled to the manually adjustable struts 106 at any time and by anyone. Once coupled, the geared-motor assemblies 302 may facilitate motorized and/or automated adjustments such as, for example, semi-continuous actuation. In some examples, the geared-motor assemblies 302 may enable motorized adjustments to be made autonomously via a companion APP running on, for example, a smartphone, a tablet, or other external computing system. Thus arranged, the spatial frame and/or system architecture may be arranged and configured to automatically adjust the motorized struts according to the prescribed treatment plan (e.g., automatically adjust the plurality of struts without patient intervention). Alternatively, or in addition to, the spatial frame and/or system architecture may be arranged and configured to require patient and/or caregiver activation to begin the process of automatically adjusting the struts according to the prescribed treatment plan. For example, the spatial frame may be arranged to intermittently auto-adjust the motorized struts at predetermined times according to the treatment plan. Alternatively, or in addition to, the spatial frame may be arranged to intermittently auto-adjust the motorized struts at selected times when convenient and/or when selected by the patient.

In some examples, the geared-motor assemblies 302 may each include an enclosure or housing 510, a coupling mechanism 520 for coupling the geared-motor assembly 302 to the strut 106, a motor 530, a torque transferring mechanism 531 (e.g., a transmission or gears for transferring rotation from the motor 530 to the strut 106), and all necessary components and circuity so that activation of the motor 530 moves the strut 106. For example, the gear-motor assemblies 302 may include one or more microprocessors, sensors such as, for example, positional sensors to monitor the length of the struts, load sensors or accelerometer for providing biomechanical feedback during bone healing and acoustic emission or vibration sensor for fault level detection in the gear train, a communication chip or antenna for facilitating communication and/or transfer of data, a power supply such as, for example, a battery, a charging circuit, etc.

Thus arranged, by utilizing geared-motor assemblies 302, motorized and/or automated adjustments of a spatial frame can be achieved. In use, the geared-motor assemblies 302 are arranged and configured to engage a manually adjustable strut 106 in an outpatient setting thus enabling the spatial frame to be operated in two different modes or configurations:
(a) a standard, manual adjustment mode where the lengths of the struts 106 can be adjusted by manual rotation of a threaded adjustment nut and (b) motorized and/or automated adjustment via the geared-motor assemblies 302.

In some examples, by arranging the geared-motor assemblies 302 as self-contained units or devices incorporating wireless, self-powered, and incorporating their own microprocessors (e.g., in some examples, the geared-motor assemblies 302 are arranged and configured as a self-contained unit including all of the necessary components and circuity to control each strut according to the prescribed treatment plan), the geared-motor assemblies eliminate the need for any external cables or wires that could snag during use and eliminate the need for incorporating a centralized master control unit onto one of the platforms of the spatial frame thereby reducing bulk and safety risk to the patient (e.g., self-containment of the control circuitry, wireless communication chip, and power source within geared-motor assemblies negate the need for cables and a centralized master control unit positioned elsewhere on the spatial frame along with any needed cables or wires).

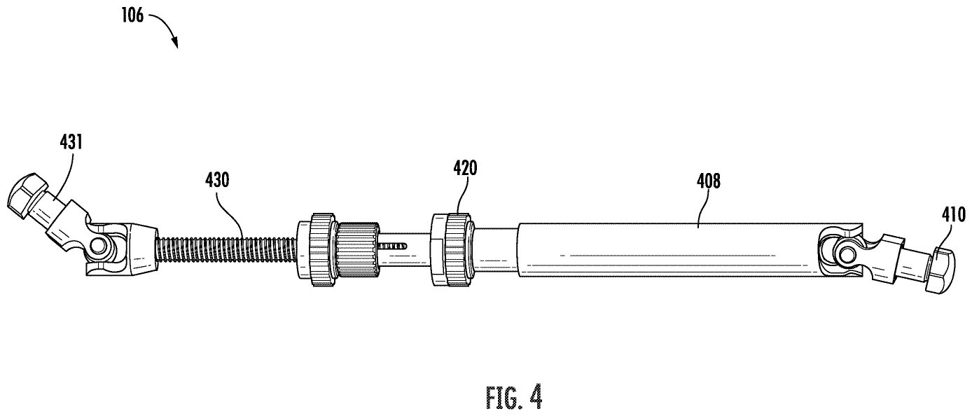

In addition, by utilizing geared-motor assemblies, existing features of the manually adjustable struts are retained. That is, with the geared-motor assemblies detached from the manually adjustable struts, operation of the struts is unaffected. For example, if the manually adjustable strut incorporates a quick adjustment feature (e.g., quick adjustment nut 508 shown in FIG. 5B) to enable manual lengthening of the strut without rotating the threaded nut or rod, such adjustment feature is retained thus enabling faster adjustment during, for example, initial setup in the operating room. Moreover, the geared-motor assemblies provide an offset motor design allowing greater application or use. For example, by incorporating an offset motor design, a shorter minimum strut length can be achieved (approximately 80 mm), which allows the struts to be used for correcting deformities in, for example, children with shorter limbs.

In some examples, when arranged in a spatial frame, the geared-motor assemblies may be arranged and configured to wirelessly exchange data, instructions, etc. with an external computing system such as, for example, a smartphone, a tablet, a computer, etc. running a companion APP. However, it is envisioned that the geared-motor assemblies may exchange data with an external computing system by any now known or hereafter developed system. For example, each of the geared-motor assemblies may include a communication interface to exchange data over a wired connection.

In some examples, the geared-motor assemblies may be water-proofed to facilitate the patient, for example, taking a shower or bath. Alternatively, or in addition to, it is envisioned that the geared-motor assemblies could be removed prior to showering and/or the spatial frame may be covered by, for example, a bag during a shower thus alleviating the necessity for water-proofing each of the geared-motor assemblies. The geared-motor assembly may also eliminate the need for sterilization since the geared-motor assemblies can be coupled to the struts in clinic.

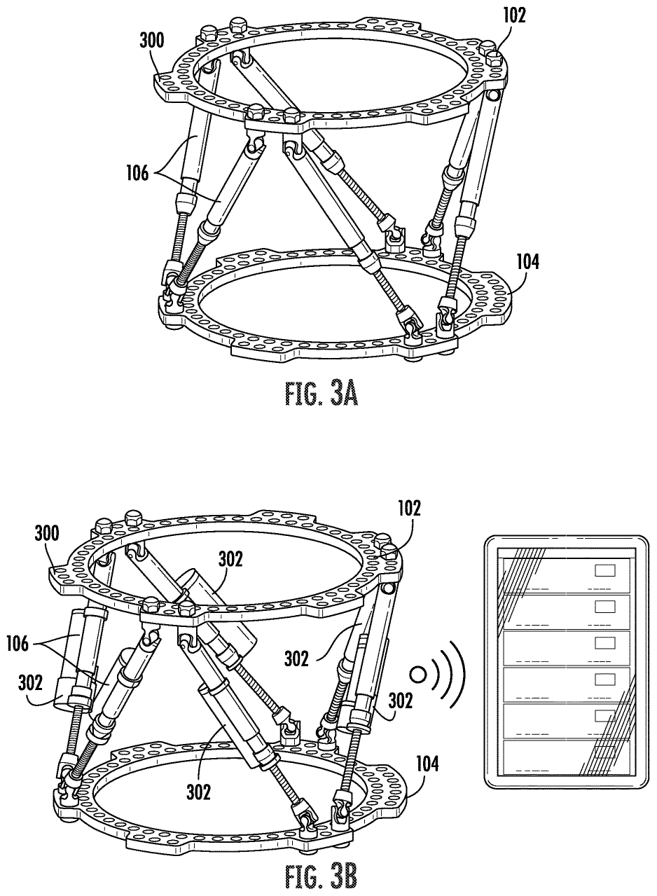

FIG. 4 shows a conventional manually adjustable strut, such as, for example, strut 106. As will be readily appreciated by one of ordinary skill in the art, the manually adjustable strut 106 includes an outer body 408 including a first joint 410 for coupling to a first platform, an internally threaded member or adjustment nut 420 coupled to the outer body 408, and an externally threaded rod or lead screw 430 including a second joint 431 for coupling to a second platform, the externally threaded rod 430 threadably engaging the adjustment nut 420. In use, the externally threaded rod 430 is constrained such that it cannot rotate relative to the outer body 408, the adjustment nut 420 is rotatably coupled to the outer body 408 but cannot translate. Thus arranged, in use, rotation of the adjustment nut 420 causes the externally threaded rod 430 to move (e.g., translate) relative to the outer body 408 to lengthen or shorten the length of the strut 106 depending on the direction of rotation. During use, the adjustment nut 420 can be manually rotated, for example, by hand or using a wrench.

In some examples, by coupling a motor to the strut 106, motorized and/or automated adjustment of the strut 106 can be achieved. For example, with reference to FIGS. 5A and 5B, the adjustment nut 420 could be modified to include teeth into an outer diameter thereof. Alternatively, or in addition to, the adjustment nut 420 could be replaced with a gear or the strut 106 could be modified to include a gear coupled to the threaded rod 430. In any event, in use, an interface is created for coupling the motor of the geared-motor assembly to the strut 106.

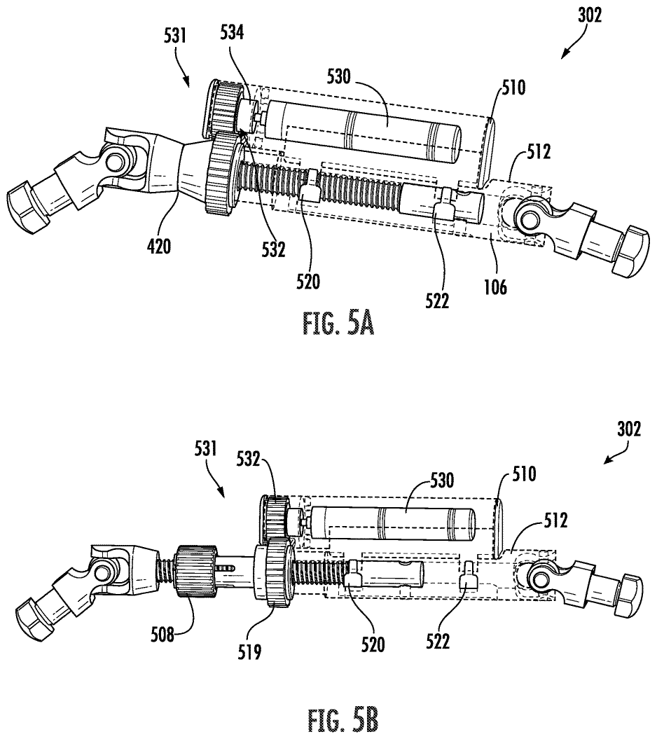

Referring to FIGS. 5A and 5B, the geared-motor assemblies 302 include a housing or enclosure 510 (terms used interchangeably herein within the intent to limit or distinguish). In use, the housing 510 is arranged and configured to enclose, or at least partially enclose, all of the components of the geared-motor assembly 302. For example, with additional reference to FIG. 6, the geared-motor assembly 302 includes a control circuit 650 (e.g., a printed-circuit board (PCB)), a microcontroller 652, a wireless communication chip, a power supply 654 such as, for example, one or more batteries, and a charging circuit 656. The electronics and the power source being housed with the motor 530 inside the housing 510. In use, when properly coupled to each of the struts 106, the geared-motor assemblies 302 facilitate motorized and/or automated adjustment of the strut 106. In addition, the geared-motor assemblies 302 may be coupled (e.g., wirelessly coupled) to an external computing system running, for example, a companion application.

In use, the geared-motor assembly 302 can be mounted to the manual struts 106 via a coupling mechanism 520, which can be arranged in any suitable mechanism now known or hereafter developed to couple or mount the geared-motor assemblies 302 to the struts 106 including, for example, clips, sleeves, magnets, straps, etc. In some examples, the coupling mechanism 520 enables easy attachment and detachment of the geared-motor assembly 302 from the strut 106 to facilitate a change in mode between manual and automated adjustment. In some examples, as shown in FIGS. 5A and 5B, the geared-motor assembly 302 may include spring loaded arms 522 arranged and configured to enable the geared-motor assembly to clip onto or engage the outer body 408 of the strut 106. In some examples, in order to better accommodate the geared-motor assembly 302, the outer body 408 of the strut 106 may be modified to include a flat surface and/or grooves 512 formed in the outer surface thereof. Thus arranged, by modifying a conventional strut to include a gear or gear teeth and optionally modifying the strut to include one or more flats and/or grooves for accommodating the geared-motor assembly 302, the modified conventional strut could be used for both manual and motorized adjustment cases.

As previously mentioned, in some examples, the geared-motor assembly 302 includes a torque transferring mechanism 531 for transferring torque from the motor 530 to the strut 106. In use, the torque transferring mechanism 531 can be any suitable mechanism now known or hereafter developed. For example, as previously described, the strut 106 and the motor 530 may include first and second gears 519, 532, respectively. In use, the first gear 519 is operatively associated with the strut 106. The second gear 532 is operatively associated with the motor 530 so that activation of the motor 530 drives (e.g., rotates) the second gear 532, which rotates the first gear 519 thereby translating the threaded rod 430 of the strut 106.

The first and second gears 519, 532 may be any suitable gear now known or hereafter developed. For example, the first and second gears 519, 532 may be pinon gears, spur gears, helical gears, a worm gear mechanism (as will be described in greater detail below), etc. Alternatively, the torque transferring mechanism 531 may be a belt drive system. In some examples, the adjustment nut 420 of the strut 106 can be modified to include an external-toothed geared surface thereby transitioning the adjustment nut 420 of the strut 106 into the first gear 519 so that the adjustment nut 420 of the strut 106 can be directly driven by the gear 532 attached to the output shaft 534 of the motor 530. Alternatively, or in addition to, the strut 106 can include a gear (e.g., the first gear 519). For example, the first gear 519 can be mounted on the threaded rod 430. In some examples, the first gear 519 can be mounted on the threaded rod 430 as a compression-fitted collar. In use, the first gear 519 engages with the second gear 532 located on the output shaft 534 of the motor 530. Thus arranged, activation of the motor 530 rotates the second gear 532, which rotates the first gear 519, which causes the strut 106 to move.

In some examples, and as previously mentioned, the geared-motor assembly 302 may include a control circuit 650. The control circuit 650 may be arranged and configured to enable autonomous, ultra-low speed movement of the strut (0.002 mm/s) and a survey of the mechanical loads exerted on the motor 530 through computation of the motor torque (DC motor current correlates with torque load on motor).

Referring to FIG. 6, as described above, the geared-motor assembly 302 includes a control circuit 650 arranged and configured as a control board or print-circuit board (PCB). As illustrated, the PCB may include a microcontroller 652, a wireless communication chip, a power supply 654 such as, for example, one or more batteries (e.g., coin cells), a charging circuit 656, and any other circuity or components needed to operate the geared-motor assemblies 302 as described herein including, for example, various surface mount devices (SMD), diodes, resistors, inductors, capacitors, etc. The control circuit 650 may be housed within the housing 510 adjacent to and extends (or runs) substantially parallel to the motor 530.

Additional information on examples of geared-motor assembly can be found in International Patent Appl. No. PCT/US2023/013011, filed on Feb. 14, 2023, entitled “Detachable Geared-Motor Assembly for Motorizing a Strut in a Spatial Frame,” which claims priority to and is a non-provisional of U.S. Patent Application No. 63/312,760, filed on Feb. 22, 2022, entitled “Detachable Geared-Motor Assembly for Motorizing a Strut in a Spatial Frame,” the entire contents of said applications being hereby incorporated by reference in their entireties herein.

It should be appreciated that while various examples of a motorized strut has been disclosed herein, it should be appreciated that one or more features of the present disclosure can be used with any suitable motorized struts now known or hereafter developed. As such, the present application should not be limited to any particular configuration or type of motorized strut unless explicitly claimed.

### II. Motorized Strut Wireless Communication

As described above, spatial frames or hexapods are used to treat deformity correction and traumatic injuries. This is accomplished using six struts that are adjusted daily to move bone segments into a desired position. As bone segments move, the process of distraction osteogenesis takes place and bone is grown by the body to bridge the gap between the segments. During this process, in many conventional systems, the struts are manually adjusted. In this case, conventional systems do not use any wired or wireless communication means. The patient and/or a caregiver adjusts each of the six struts to a particular length each day based on an adjustment schedule provided in a treatment plane (e.g., in a hardcopy form or digitally).

While some conventional systems provide automation capabilities (as discussed above) to its spatial frames, these systems rely on wired connections for its components for the purposes of communications. The strut adjustment schedule is typically generated on a computer and loaded to the control module of the spatial frame through a USB connection. The control module mounts to the spatial frame and is stored in the control module's memory. Wires connect the control module to each of the six motors that drive the struts so that the control module can send power to the motors to complete a particular adjustment. This makes the entire frame very heavy and bulky. Moreover, because wires have to run, either through the frame and/or on the outside of the frame, the entire system may be prone to breakdowns (e.g., wires bending, breaking, etc.), leading to incorrect adjustments as well as added pain to the patient.

In some examples, the present disclosure relates to a spatial frame that may be configured to wireless connect its components without requiring any wired connections. The present disclosure may be configured to include one or more wireless communication modules (e.g., BluetoothTM, near field communication (NFC), etc.) that may be configured to be communicatively coupled to the frame's motorized struts and/or any other components (each of which may be equipped with a wireless communication module of its own). The wireless communication module may be configured receive any adjustment plans and/or changes to the plan(s) via wireless communication from one or more external devices and store them in the memory coupled to a processor of the spatial frame. The processor may be configured to execute one or more instructions related to adjustment of one or more motorized struts and cause their transmission, via the wireless communication module, to such struts for the purposes of adjustment. Each strut's wireless communication module may be configured to receive such instructions and trigger operation of its motor to perform adjustment. A clear advantage of the present disclosure is an absence of wiring that is required (by the conventional systems) to connect each strut's motor to the main control module, which in turn, unclutters the entire system as well as reduces possibilities of a breakdown, such as, for example, through bending, breaking, etc. of the wires.

As discussed above in connection with FIGS. 5A-6, each strut of the motorized spatial frame may be configured to include a PCB, a battery, and a motor. As can be understood, each of and/or some and/or all the struts may include all three of these components and/or some of these components. For instance, one or more batteries and/or PCB components may be housed on the ring of the motorized spatial frame and/or anywhere else on the spatial frame. The battery may be configured to provide power to the motor. The motor may cause lengthening and/or shortening of one or more struts by turning one or more gears that interact with an internally threaded member coupled to the threaded rod of the strut.

In some examples, one or more PCBs (and/or any other processing circuits) in the spatial frame's motor modules may include one or more wireless transceivers that may be configured to execute communication functions one or more wireless communication protocols, such as, for example, Bluetooth™, Bluetooth™ Low Energy (BLE), Near Field Communication (NFC), and/or any other protocols. The communication functions may include receiving and/or transmitting one or more signals between one or more motor modules and/or external devices.

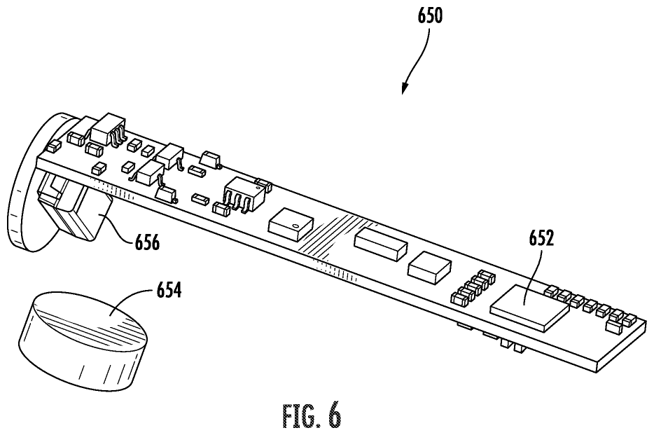

FIG. 7 is a block diagram of a system 750 that may be used with a spatial frame (as, for example, is shown in FIGS. 1-6), according to some examples of the present disclosure. The system 750 may be configured to include a spatial frame system 700 that may be configured to be communicatively coupled via a network 703 to an external device 701. The network 703 may include one or more of local area networks (LAN), wide area networks (WAN), infrared, radio, Bluetooth, Wi-Fi, point-to-point (P2P) networks, telecommunication networks, cloud communication, and the like. The device 701 may include, for example, but not limited to, a mobile telephone, a tablet, a laptop, a personal computer, a personal digital assistant, and/or any other device.

The spatial frame system 700 may include motor modules 702(a, b, c, d, e, f). For ease of illustration only, FIG. 7 illustrates four modules 702 (e.g., 702a, 702b, 702c, and 702f). Each motor module 702 may be configured to include its respective processor 704, memory 706, wireless module 708, and motor components 710 (e.g., electro-mechanical components and circuitry, sensor(s), power source(s), etc.). For example, the module 702 may include a processor 704a, a memory 706a, a wireless module 708a, and motor components 710a. Optionally, the spatial frame system 700 may also include a separate control module 712. The components 704-708 may be communicatively coupled to one another within the respective motor module 702.

The processor 704 may be configured to receive instructions for execution of various functionalities associated with operation of the motor module 702, including, but not limited to, the motor components 710. The processor 704 may receive such instructions via the wireless module 708. The instructions may be received and stored in the memory 706, where the processor 704 may query the memory 706 for its operational instructions. Alternatively, or in addition, the processor 704 may receive operational instructions on-the-fly, which may allow for dynamic adjustment of operations of each motor module 702.

As stated above, the memory 706 may be configured to store various data associated with operation of motor module 702. The data may include the treatment plan that may be transmitted from the external device 701. The treatment plan may be stored by memory 704 of each motor module 702.

In some examples, the motor modules 702 may be independent modules and may be communicatively coupled to one another to allow for sharing of various data associated with each motor module 702. This may ensure that all motor modules 702 are operating in accordance with the treatment plan and/or goals set by such plan. The data shared via such communications may be used for monitoring of operations of each motor module 702 by other motor modules 702, external devices 701, and/or optional control module 712. The data may also be used to perform adjustments to the operation of one or more motor modules 702.

The wireless modules 708 of motor modules 702 may be configured to execute various receiving and/or transmitting functionalities to receive and/or transmit signals between modules 702 and/or external devices 701 and/or optional control module 712 (which, for example, may also include its own communication component). The modules 708 may be used for transmission and/or receiving of operational data associated with operation of each motor module 702. The transmission/receiving of such data may be accomplished wirelessly, such as, for example, using Bluetooth™, Bluetooth™ Low Energy (BLE), Near Field Communication (NFC), and/or any other protocols, and/or any combination of protocols.

In some examples, one motor module 702 (e.g., module 702a), may be configured to serve as a main module, and may be communicatively coupled to other motor modules 702 (e.g., modules 702(b-f)), that may be configured to serve as secondary modules. The secondary modules 702 may or might not be communicatively coupled to other secondary modules 702. The main module 702a may be configured to receive operational instructions (e.g., treatment plan and/or any updates thereof) from the device 701, store them in its memory 704a, monitor operation of each of the secondary modules 702(b-f), and provide operational instructions (e.g., strut adjustment instructions) to the secondary modules 702(b-f) via its wireless module 708a. The wireless module 708a may be communicatively coupled to each of the modules 708(b-f) as well as the device 701 via the network 703.

The main module 702a may be configured to query, ping, transmit heartbeat messages to, etc. each of the secondary modules 702(b-f) during operation to obtain operational status of each the secondary modules 702(b-f). Alternatively, or in addition, each of the secondary modules 702(b-f) may be configured to automatically report their respective operational statuses to the main module 702a. The processor 704a of the main module 702a may be configured to analyze data received from the secondary modules 702(b-f) to determine whether adjustments of operation of one or more of such secondary modules 702(b-f) may be necessary (e.g., based on the healing data, pressure data, load data, etc.).

In some examples, more than one main module 702 (e.g., 702a and 702b) may be used for communicating with a group of secondary modules 702 (e.g., module 702a may control modules 702(c-e) and module 702b may control modules 702(f-g)). The main modules 702a and 702b may be communicatively coupled to one another and may process various data received from secondary modules 702 and/or from each other to determine optimal operation of the spatial frame system 700.

In other examples, the control module 712 may be configured to serve as a main communication and control module. The module 712 may be configured to be communicatively coupled (e.g., wirelessly) to each of the motor modules 702. The control module 712 may be configured to receive operational instructions (e.g., treatment plan) from the external device 701 via the network 703 and store it in its memory. Based on the received treatment plan, the module 712 may transmit (e.g., wirelessly) appropriate operational instructions to each of the modules 702 for execution of adjustments of motorized struts. The module 712 may be configured to monitor operation of each of the modules 702, process data received as a result of the monitoring, generate modifications (if necessary) to the motorized strut adjustment instructions and transmit same to one or more modules 702 for execution. Each of the modules 702 may be configured to report to the control module 712 on the status of their respective operations, which may include healing data, load data (e.g., determined based on a current pull from their respective power sources), pressure data, etc. The reporting may be executed automatically, periodically, using a predetermined schedule, and/or upon request from the control module 712.

In some examples, the spatial frame system 700 may be configured to be equipped with authentication mechanism to prevent unauthorized use of the system 700. For example, to access the system 700 using the device 701, the user (e.g., doctor, patient, etc.) may be requested to input various authentication credentials (e.g., username and password, a verification key, a QR code, a barcode, etc.) using a user interface of the device 701. The authentication credentials may be associated with a particular treatment plan, user (e.g., patient, doctor, etc.), and/or any other information related to the treatment, user, etc.

Once the authentication credentials are verified, the user may be allowed to login into the system, view operational data, upload operational instructions (e.g., treatment plan), and/or perform any other functions. Use of authentication mechanisms may be configured to prevent submission of incorrect or fake treatment plans.

By logging in into the system, the device 701 and one or more wireless modules 708 and/or control module 712 may be configured to be paired with the device 701 to permit secure communications between the device 701 and the system 700. For pairing purposes, the system 700 may be configured to include one or more pairing buttons (e.g., incorporated into one or more modules 702 and/or control module 712) that may be pressed for pairing purposes. In some non-limiting examples, NFC may be used to “wake” one or more motor modules 702 and force it to enter the pairing mode automatically. NFC may be used to enhance the security of the Bluetooth™ connection, being used as an out of band (OOB) security check (e.g., in order to establish a Bluetooth™ connection, NFC may be used as well so that to pair, the device 701 may have to be within a predetermined distance (e.g., couple of inches) of the pairing motor module 702).

In some examples, only one or primary motor module 702 (and/or control module 712) may be configured to be paired with the device 701, whereby other or secondary motor modules 702 communicate with the paired primary motor module 702 without being paired to the device 701. Each of the secondary modules 702 may be configured to be paired with the primary module 702. The pairing operations may need to be completed and/or verified prior to implementation of the treatment plan. Alternatively, or in addition, all and/or some motor modules 702 may be paired with the device 701. Each of these options may be configured to provide an ability to individually control each motor module 702 and/or control a group and/or all of motor modules 702.

In some examples, each of the modules 702 may be configured to pre-paired with one another during production, which may reduce time associated with pairing of the modules 702 during fitting of the spatial frame on the patient. The pre-pairing of the modules 702 may include loading appropriate authentication information, encryption keys, etc. to the modules' memory, whereby upon being powered on, the modules 702 may automatically be communicatively coupled to one another.

In some examples, a treatment plan may be directly loaded into one or more modules 702 (and/or control module 712). This may avoid a need for the external device 701, thereby allowing the spatial frame system 700 to be completely autonomous. Any adjustments to the treatment plan may be determined by one or more processors 704 (and/or control module 712) based on various data that may be collected during operation of the system 700.

In some examples, the device 701, once paired to the frame 700 and/or any of its modules, may be configured to periodically determine that an update to the previously loaded treatment plan may be necessary. In that case, the device 701 may be configured to push such updates to the frame 700/its modules. The pushed updates may be processed by the frame 700/its modules and implemented accordingly.

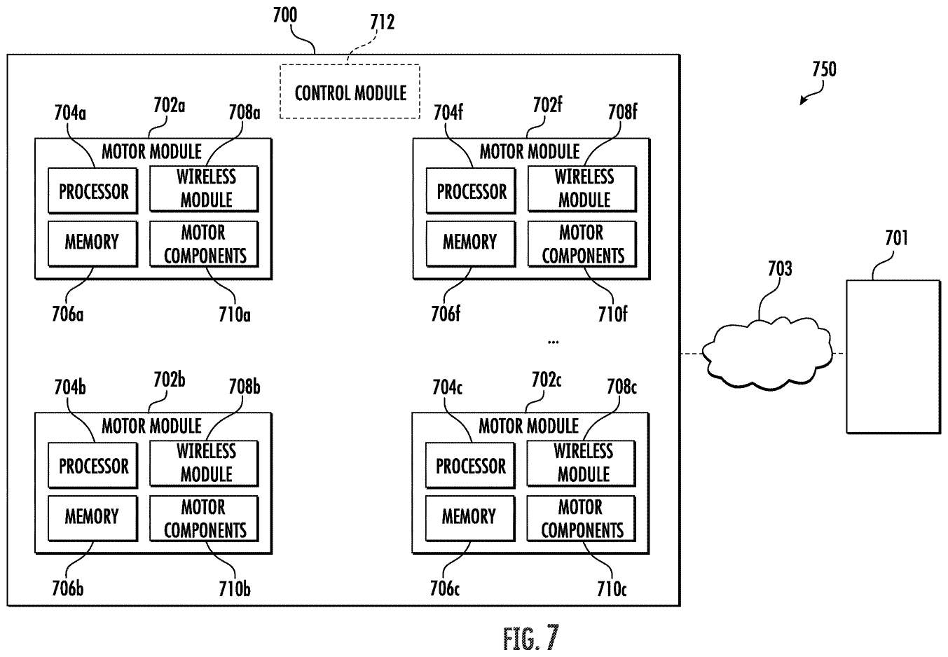

FIG. 8 illustrates a process 800 for executing wireless communications using a spatial frame, according to some examples of the present disclosure. The process 800 may be executed by the system 750, including, but not limited to, one or more motor modules 702 and/or optional control module 712 and/or external device 701, where communications between these components may be accomplished using various wireless communications protocols (e.g., BluetoothTM, BLE, NFC, etc.).

At 802, a prescribed treatment plan may be received. The treatment plan may be generated for a particular patient and patient's medical condition. The treatment plan may be loaded into the device 701 and/or one or more processors 704 and/or memories 706 (and/or control module 712, if equipped). The treatment plan may also be loaded during the manufacturing process of the spatial frame system 700. During the process, the modules 702 may also be configured to be pre-paired with one another. Alternatively, or in addition, the pairing process may occur during powering of motor modules 702. The pairing may be executed between one or more modules 702 and/or between one or more modules 702 and/or device 701. As discussed, there may be various options for pairing and/or controlling of the system 700 may be used (e.g., one module 702 may be a primary module configured to communicated with other or secondary modules 702 as well as the device 701; more than one primary modules 702 may be used, each of which may be configured to communicate with a group of secondary modules 702 and the device 701; each module 702 may be individually paired with the device 701; and/or the device 701 is not used and the system 700 may be configured to operate autonomously).

At 804, a determination may be made whether all modules 702 are appropriately paired (as discussed above). If one or more modules 702 have not be paired, either with a primary module 702, and/or other modules 702, and/or device 701, and/or any other device, as may be required for operation of the spatial frame 700, the pairing process may continue, at 806, until confirmation of pairing of all modules is determined and/or received (e.g., each motor module 702 may be equipped with a LED light that may turn green upon confirmation of pairing).

If, at 804, all modules 702 are appropriately paired, the process 800 may be configured to continue with transmission of one or more operation instructions (as may be defined in the treatment plan) to each motor module 702, at 808 to perform adjustments of motorized struts in accordance with the treatment plan (or modified treatment plan), at 810.

At 812, it may be determined that an adjustment to the treatment plan may be necessary. For example, an analysis of data that may be collected by one or more processors 704 of modules 702 may result in a determination that there is an uneven loading on one or more motorized struts as a result of execution of the treatment plan. If so, a modification of the plan may be determined, at 814, and executed accordingly, at 816. The system 700 may continue monitoring execution of the plan or the modified plan.

In some examples, the present disclosure may be manufactured and/or sold as a pre-paired kit (e.g., struts, motor modules, etc.), where each motor module may be pre-paired during manufacture. One motor module may be selected as a primary motor module and may connect to other motor modules. Alternatively, or in addition to, separate connections by each motor module may be established and used. The treatment plan may be delivered to the system 700 (e.g., to the primary module and/or each individual module) via a wired and/or wireless connection.

In some examples, communication hub (e.g., incorporated into the control module 712) may be used for handling wireless communications between the motor modules and one or more external devices 701. This hub may also include a user interface and/or other means for displaying device status. Connections may be established and secured using various means, e.g., an access code, a username and password, etc. One or more external devices 701 may be equipped with an app that may be used for setup of the spatial frame, obtaining status of treatment, perform adjustments of one or more motorized struts, etc.

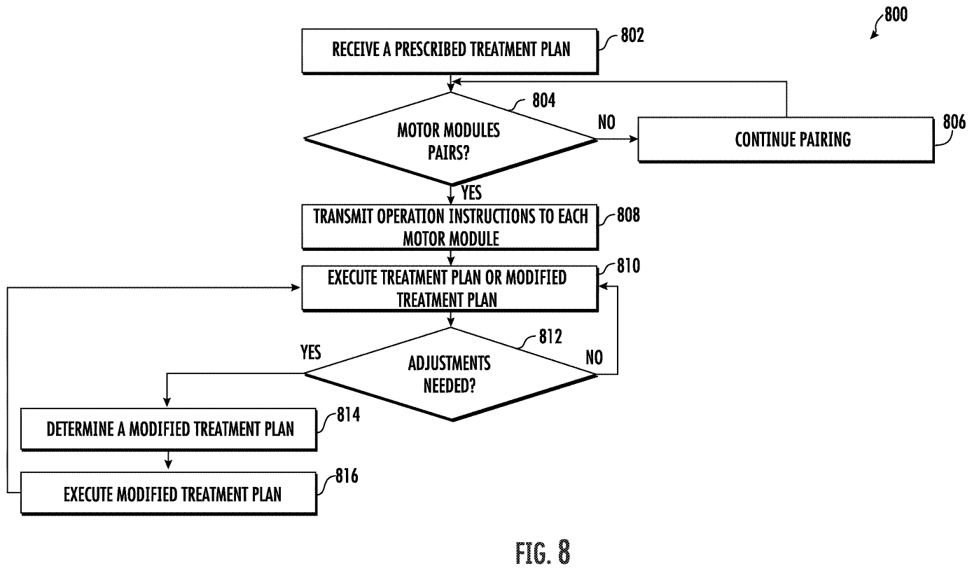

FIG. 9 illustrates a process 900 for executing a computing update, e.g., a software update, a firmware update, etc., operating on a spatial frame, according to some examples of the present disclosure. The process 900 may be executed by the system 750 (as shown in FIG. 7), including, but not limited to, one or more motor modules 702 and/or optional control module 712 and/or external device 701, where communications between these components may be accomplished using various wireless communications protocols (e.g., Bluetooth™, BLE, NFC, etc.). While the following discussion of the process 900 is presented in connection with a firmware update to firmware installed on one or more struts (and/or all struts) of the spatial frame, as can be understood, the process 900 may be applicable to any type of computing update. The process 900 may also be executed on specifically selected strut(s) but not other struts, and/or on all struts. Further, the process 900 may be executed on all struts simultaneously and/or sequentially (one strut after the other). Alternatively, or in addition, the process 900 may be executed for updating a firmware on one of the struts, which may be designated as a primary strut, where the primary strut may be configured to push the firmware update to other struts, which may be designated as secondary struts. Any strut may be designated as primary for the purposes of the firmware update. Moreover, the firmware update may likewise be performed for the control module 712.

At 902, a firmware update may be received. In some examples, the firmware update may be an update to one or more software functionalities associated with software used by the strut(s) to perform one or more operations outlined in the prescription plan. Alternatively, or in addition, the firmware update may be an update to the entire operating system used by the strut(s). As can be understood, any other types of updates may be performed.

In some examples, the firmware update may be received by the firmware update may be uploaded to a website, with which external device 701 may be configured to communicate. The website may be configured to be administered by a manufacturer of the spatial frame and/or by any other entity. The external device 701 may be configured to download the firmware update from the website to its internal storage and/or memory. Alternatively, or in addition, the firmware update may be directly downloaded to the internal storage and/or memory of the external device 701.

At 904, the external device 701 may be configured to compare the downloaded firmware update to the firmware installed on one or more struts of the spatial frame. This may, for instance, be accomplished through comparison of version identifiers associated with firmware that is currently installed on one or more struts and the downloaded firmware update. In some examples, the external device 701 may have previously stored an identifier of the version of the firmware installed on one or more struts. Alternatively, or in addition, the external device 701 may communicate, via network 703, with one or more or each of the struts' motor modules 702 and/or control module 712 to determine version of firmware installed on each strut. In some examples, the external device 701 may communicate with a primary motor module 702 (e.g., motor module 702a), which in turn, may poll other motor modules 702 (e.g., motor modules 702b-f) to determine their respective versions of firmware and communicate version information for all struts of the spatial frame to the external device 701.

If at 906, the external device 701 determines that the version of firmware update it received is not more recent than the version of firmware installed on strut(s) (one or more or all), it may, optionally, terminate the firmware update process, at 926. Otherwise, the process 900 may proceed to determine whether or not the received firmware update is critical, at 908. A critical update may be an update that is required for proper operation of the spatial frame whether or not in accordance with the prescription plan. A critical update may also be an update that may result in conserving of power in the power source (e.g., battery) of the spatial frame. A critical update may also be an update that protects integrity of the operational system of one or more or all struts (and/or optional control module 712).

If the firmware update is not critical, at 908, then the firmware update process may be delayed, at 910. For example, the firmware update process of any non-critical updates may be delayed until all critical firmware (and/or other) updates have been completed. Moreover, the firmware update process of any and/or all of the struts may be delayed until full and/or partial treatment is completed and/or activity of any and/or all of the struts has been paused or stopped (e.g., during no strut adjustment break in the prescription plan). Upon completion of the delay, the process 900 may proceed to 912 for processing of firmware update, as described below.

If the update is determined to be critical, at 908, the update process 900 may proceed regardless of whether a treatment plan is or is not underway. This may allow firmware updates to be immediately installed so that the spatial frame and all of its struts are functioning properly.

At 912, firmware update may be downloaded and transferred to one or more struts, at 914. In some examples, the firmware update may be downloaded by the external device 701 to its storage location from an external source (e.g., website). Once downloaded, the external device 701 may transfer the firmware update to one or more struts, and in particular to memory 706 of the motor module 702. For instance, the external device 701 may select a strut (e.g., designated as a primary strut) and communicate with its motor module (e.g., motor module 702a) using motor module's wireless module (e.g., wireless module 708a) via network 703. The external device 701 may then transfer the downloaded firmware update to the memory of the motor module (e.g., memory 706a). The memory of the motor module that is receiving the firmware update may be memory and/or specifically designated memory that is not actively used by the motor module to execute instructions in the prescription plan. This way, any interruptions to use of available memory that may be associated with transfer of firmware update are avoided, thereby avoiding interruptions to the movements defined by the prescription plan being performed by the struts. For example, the firmware update may be transferred to a secondary flash memory and/or any other type of memory of the motor module.

Alternatively, or in addition, if the external device 701 has directly received the firmware update, it may transfer the update to one or more struts. As can be understood, the external device 701 may transfer the firmware update to selected strut or struts and/or all struts.

At 916, the processor (e.g., processor 704a) of the motor module that has received the firmware update may be configured to authenticate the source of the firmware update. For example, the strut's processor (e.g., processor 704a) may verify a digital signature associated with the firmware update. This process may involve use of public-private key pair protocol and/or any other verification protocol that may be executed by the processor using via the wireless module (e.g., module 708a) and the network 703.

Upon verification of the source of the firmware update, the processor of the motor module may assess integrity of the firmware update, e.g., to ensure that it is not corrupted, free of errors, etc. This may, for instance, be accomplished using a cyclic redundancy check (CRC), an MD5 check, and/or any other procedures.

At 918, the processor (e.g., processor 704a) of the motor module may determine whether its strut is currently active. This may be accomplished by determining whether any of the motor components (e.g., motor components 710a) are currently being in motion, such as, through determination of whether any current is being drawn from the power source and/or through any other ways.

If, at 918, the strut is active, the processor (e.g., processor 704a) may delay installation of the update, at 920. Otherwise, the processor may execute the installation of the firmware update, at 922. Subsequent to the execution of the installation, the processor may, optionally, be configured to reboot the strut's computing system and perform requisite checks to ensure that firmware update has been installed.

At 924, a determination may be made whether firmware update on other struts in the spatial frame may be needed. This may be accomplished by each strut's motor module (e.g., motor modules 702b-f) communicating with the external device 701 via the network 703 to determine whether operations 912-922, if any, may need to be performed. Alternatively, or in addition, as discussed herein, one of the struts may be designated as a primary strut (and hence, its motor module, e.g., motor module 702a) and may be configured to control transfer and/or installation of firmware update to other secondary struts. In this scenario, source authentication and/or verification of the firmware update may be skipped by the secondary struts, because the primary strut may have authenticated the source of and verified the firmware update.

Once firmware on struts that may have required the firmware update has been appropriately update, the process may terminate, at 926.

As stated above, one of the advantages of the present disclosure is use of wireless communications between motorized modules and/or any external devices. This avoids use of wired connections on the spatial frame, thereby reducing an amount of space that is used by the frame, wires, and/or its other components, decreasing weight of the spatial frame, and preventing wire-snagging on objects around the patient. Additionally, wireless transmission of data (including treatment plan) to the spatial frame and between its components may be advantageous in that the patient might not have to come to a doctor's office for adjustments to the frame's motorized struts. The doctor may use an external device to monitor the treatment, perform adjustments and/or control operation of the motorized frame. Further, any updates to firmware, software, etc. of motorized modules may be seamlessly executed. Moreover, any critical updates may be pushed to the computing systems of motorized modules without delay ensuring proper operation of the spatial frame.

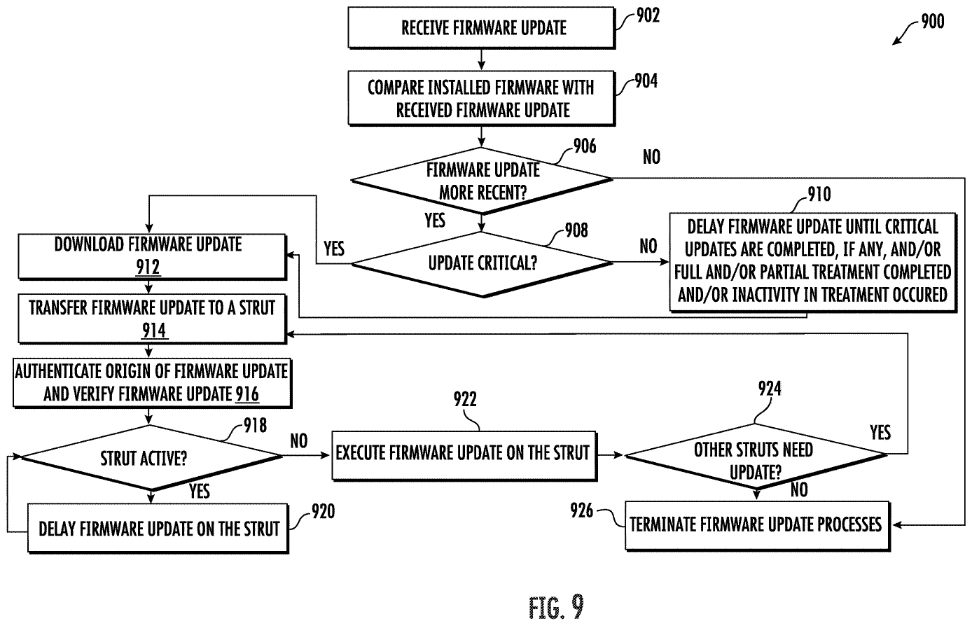

FIG. 10 illustrates an exemplary computing apparatus 1000, according to some examples of the present disclosure. The apparatus 1000 may be a computing device that may be communicatively coupled with a spatial frame and/or device communicatively coupled to the spatial frame such as, spatial frame shown in FIGS. 3a-6. The apparatus 1000 may be a computer in the form of a smart phone, a tablet, a notebook, a desktop computer, a workstation, or a server. The apparatus 1000 can combine with any suitable example of the systems, devices, and methods disclosed herein. The apparatus 1000 can include processor(s) 1010, a non-transitory storage medium 1020, communication interface 1030, and a display 1035. The processor(s) 1010 may include one or more processors, such as a programmable processor (e.g., a central processing unit (CPU)). The processor(s) 1010 may include processing circuitry to implement spatial frame circuitry 1015.

The processor(s) 1010 may include memory such as flash memory to contain program code for execution by the processor(s) 1010. In some implementations, the processor(s) 1010 may have random access memory to contain a copy of code from flash memory or read only memory to facilitate faster execution of code. In some implementations, the processor(s) 1010 may include cache to contain data for faster calculations or execution. In some implementations, the processor(s) 1010 may include spatial frame circuitry 1015, which may include a user interface manager 1017. The user interface manager 1017 may function as a state machine controlled by keypad inputs, internal events or alarms, boundary conditions, exceptions, and supervisory input to the user interface manager 1017. The user interface manager 1017 may process button presses and may update a main screen on the display 1035 reflecting the state of the application.

Motor controller commands may be executed automatically and/or upon the user's actions via button presses, system states, and error conditions. Further, the user interface manager 1017 may implement alerts, warnings, and notifications and display the alerts, warnings, and notifications via the display 1035. The user interface manager 1017 may also include code to handle the user's response to alerts, warnings, and notifications.

The processor(s) 1010 may operatively couple with a non-transitory storage medium 1020. The non-transitory storage medium 1020 may store logic, code, and/or program instructions executable by the processor(s) 1010 for performing one or more instructions including the spatial frame circuitry 1025. The non-transitory storage medium 1020 may include one or more memory units (e.g., fixed and/or removable media or external storage such as electrically erasable programmable read only memory (EEPROM), a secure digital (SD) card, random-access memory (RAM), a flash drive, solid-state drive, a hard drive, and/or the like). The memory units of the non-transitory storage medium 1020 may store logic, code and/or program instructions executable by the processor(s) 1010 to perform any suitable implementation of the methods described herein. For example, the processor(s) 1010 may execute instructions such as instructions of spatial frame circuitry 1025 causing one or more processors of the processor(s) 1010 to communicate user commands to the spatial frame 300 (as shown in FIGS. 3a-6) and/or to communicate events, alerts, operation parameters for the spatial frame 300, and configurations.

The panels 1028 may define graphical user interfaces for display of information and for receiving input parameters or configurations from a user. The configuration file 1029 may include user selected parameters such as, for example, adjustment rates, motor pull, current pull, etc.

The processor(s) 1010 may couple to a communication interface 1030 to transmit the data, code, or commands to and/or receive data, code, or commands from one or more external devices (e.g., a terminal, display device, a smart phone, a tablet, a server, or other remote device). The communication interface 1030 includes circuitry to transmit and receive communications through a wired and/or wireless media such as an Ethernet interface, a wireless fidelity (Wi-Fi) interface, a Bluetooth interface such as a Bluetooth Low Energy (BLE) interface, a cellular data interface, and/or the like. In some examples, the communication interface 1030 may implement logic such as code in a baseband processor to interact with a physical layer device to transmit and receive wireless communications from the spatial frame 300. For example, the communication interface 1030 may implement one or more of local area networks (LAN), wide area networks (WAN), infrared, radio, Bluetooth, Wi-Fi, point-to-point (P2P) networks, telecommunication networks, cloud communication, and the like.

The processor(s) 1010 may couple to a display 1035 to display panels 1028 for a user interface and/or other user interface items such as a message or notification via, graphics, video, text, and/or the like. In some examples, the display 1035 may include a display on a terminal, a display device, a smart phone, a tablet, a server, or a remote device.

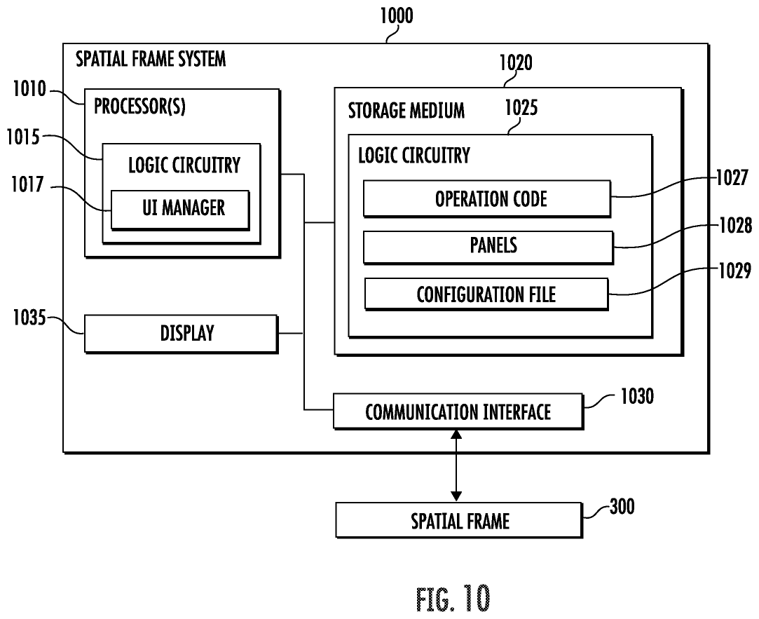

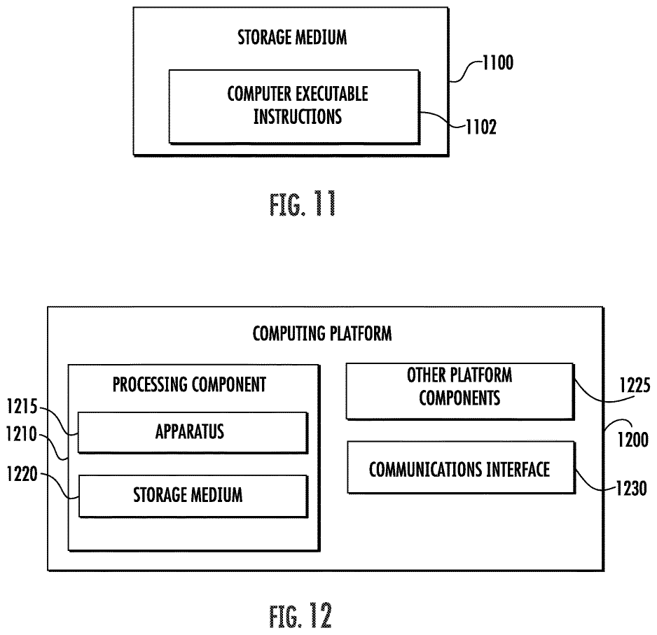

FIGS. 11-12 illustrate example implementations of a storage medium and computing platform for a spatial frame in accordance with one or more features of the present disclosure. FIG. 11 illustrates an example of a storage medium 1100 to store spatial frame logic. Storage medium 1100 may include an article of manufacture. In some examples, storage medium 1100 may include any non-transitory computer readable medium or machine-readable medium, such as an optical, magnetic or semiconductor storage. Storage medium 1100 may store various types of computer executable instructions 1102, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 12 illustrates an example computing platform 1100. In some examples, as shown in FIG. 12, the computing platform 1200 may include a processing component 1210, other platform components or a communications interface 1230. According to some examples, computing platform 1200 may be implemented in a computing device such as a server in a system such as a data center or server farm that supports a manager or controller for managing configurable computing resources as mentioned above. Further, the communications interface 1230 may include a wake-up radio (WUR) and may be capable of waking up a main radio of the computing platform 1200.

According to some examples, processing component 1210 may execute processing operations or logic for apparatus 1215 described herein such as the spatial frame logic circuitry 1015, and 1025 illustrated in FIG. 10. Processing component 1210 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 1220, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 1225 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 1230 may include logic and/or features to support a communication interface. For these examples, communications interface 1230 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012. Network communication may also occur according to one or more OpenFlow specifications such as the OpenFlow Hardware Abstraction API Specification. Network communications may also occur according to InfiniBand Architecture Specification, Volume 1, Release 1.3, published in March 2015.

Computing platform 1200 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 1200 described herein, may be included, or omitted in various implementations of computing platform 1200, as suitably desired.

The components and features of computing platform 1200 may be implemented using any combination of discrete circuitry, ASICs, logic gates and/or single chip architectures. Further, the features of computing platform 1200 may be implemented using microcontrollers, programmable logic arrays and/or microprocessors or any combination of the foregoing where suitably appropriate. It is noted that hardware, firmware and/or software elements may be collectively or individually referred to herein as “logic.”

It should be appreciated that the exemplary computing platform 1200 shown in the block diagram of FIG. 12 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in implementations.

One or more features of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores,” may be stored on a tangible, machine readable medium and supplied to various customers or manufacturing facilities to load into the fabrication machines that actually make the logic or processor.

The foregoing description has broad application. While the present disclosure refers to certain implementations, numerous modifications, alterations, and changes to the described implementations are possible without departing from the sphere and scope of the present disclosure, as defined in the appended claim(s). Accordingly, it is intended that the present disclosure not be limited to the described implementations. Rather these implementations should be considered as illustrative and not restrictive in character. All changes and modifications that come within the spirit of the present disclosure are to be considered within the scope of the disclosure. The present disclosure should be given the full scope defined by the language of the following claims, and equivalents thereof. The discussion of any implementation is meant only to be explanatory and is not intended to suggest that the scope of the disclosure, including the claims, is limited to these implementations. In other words, while illustrative implementations of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counter-clockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular implementations. Such terms are not generally limiting to the scope of the claims made herein. Any implementation or feature of any section, portion, or any other component shown or particularly described in relation to various implementations of similar sections, portions, or components herein may be interchangeably applied to any other similar implementation or feature shown or described herein.

It should be understood that, as described herein, an “implementation” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated implementations are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one implementation” of the present disclosure are not intended to be interpreted as excluding the existence of additional implementations that also incorporate the recited features.

In addition, it will be appreciated that while the Figures may show one or more implementations of concepts or features together in a single implementation of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one implementation can be used separately, or with another implementation to yield a still further implementation. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “includes” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one,” “one or more,” and “and/or” as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more implementations or configurations for the purpose of streamlining the disclosure. However, it should be understood that various features of the certain implementations or configurations of the disclosure may be combined in alternate implementations or configurations. Moreover, the following claims are hereby incorporated into this Detailed Description by this reference, with each claim standing on its own as a separate implementation of the present disclosure.

1. A spatial frame apparatus, comprising:
a frame;
at least two motor modules configured to be supported by the frame, each motor module comprising:
one or more motorized struts;
one or more motors configured to cause movement of the one or more motorized struts in one or more directions in accordance with one or more treatment plans;
wherein at least one motor module in the at least two motor modules is configured to be wirelessly coupled to at least another motor module in the at least two motor modules;
wherein each motor module in the at least two motor modules comprises one or more processing components communicatively coupled to at least one of: the one or more motorized struts and the one or more motors; and
wherein the one or more processing components are configured to execute at least one adjustment of the one or more motorized struts based on the one or more treatment plans and in response to one or more wireless signals received from at least one of: at least one motor module in the at least two motor modules and one or more external devices communicatively coupled to at least one motor module in the at least two motor modules.

2. The apparatus of claim 1, wherein each motor module in the at least two motor modules includes one or more wireless communication components configured to wirelessly communicate with at least one of: at least another motor module in the at least two motor modules and the one or more external devices.

3. The apparatus of claim 2, wherein the one or more wireless communication components are configured to be paired with at least one of: at least another wireless communication component in the one or more wireless communication components and with the one or more external devices.

4. The apparatus of claim 2, wherein the one or more wireless communication components are configured to wirelessly communicate with at least another wireless communication component in the one or more wireless communication components using one or more communication protocols; the one or more communication protocols comprising at least one of the following: Bluetooth™ communication protocol, Bluetooth™ Low Energy (BLE) communication protocol, near field communication (NFC) protocol, and any combination thereof.

5. (canceled)

6. The apparatus of claim 1, wherein at least one motor module in the at least two motor modules is selected as a primary motor module and at least another motor module in the at least two motor modules is selected as a secondary motor module.

7. The apparatus of claim 6, wherein the primary motor module is configured to transmit one or more instructions to the secondary motor module for execution of the treatment plan.

8. The apparatus of claim 6, wherein the one or more external devices are wirelessly coupled to the primary motor module, wherein the primary motor module is configured to receive one or more instructions from the one or more external devices and transmit the one or more received instructions to the secondary motor module.

9. The apparatus of claim 8, wherein the one or more external devices are configured to execute at least one of: monitoring, controlling, and adjusting operation of at least one motor module in the at least two motor modules in response to the one or more wireless signals.

10. The apparatus of claim 9, wherein the one or more motors cause the one or more motorized struts to execute the at least one adjustment in accordance with the one or more treatment plans, one or more parameters associated with the at least one adjustment include at least one of the following: a direction of the at least one adjustment of the one or more motorized struts, a length of the at least one adjustment of the one or more motorized struts, an angle of the at least one adjustment of the one or more motorized struts, a time for the at least one adjustment of the one or more motorized struts, one or more parameters for changing positioning of the one or more motorized struts, and any combination thereof.

11. (canceled)

12. The apparatus of claim 1, wherein at least one motor module in the at least two motor modules is configured to receive one or more firmware updates using the one or more wireless signals.

13. The apparatus of claim 12, wherein the one or more processing components:
authenticate a source of the one or more firmware updates;
verify the one or more firmware updates; and
install, upon authentication and verification of the one or more firmware updates, the one or more firmware updates on an operating computing system of at least one motor module in the at least two motor modules.

14. The apparatus of claim 13, wherein the one or more processing components perform at least one of: authentication of the source of the one or more firmware updates, verification of the one or more firmware updates, and installation of the one or more firmware updates without interruption of execution of the at least one adjustment of the one or more motorized struts based on the one or more treatment plans.

15. A method, comprising:
providing a spatial frame apparatus comprising:
a frame;
at least two motor modules configured to be supported by the frame, each motor module comprising:
one or more motorized struts;
one or more motors configured to cause movement of the one or more motorized struts in one or more directions in accordance with one or more treatment plans;
wherein at least one motor module in the at least two motor modules is configured to be wirelessly coupled to at least another motor module in the at least two motor modules;
wherein each motor module in the at least two motor modules comprises one or more processing components communicatively coupled to at least one of:
the one or more motorized struts and the one or more motors;
receiving, using the one or more processing components, one or more wireless signals representative of at least one adjustment of the one or more motorized struts based on the one or more treatment plans; and
executing, using the one or more processing components, the at least one adjustment of the one or more motorized struts based on the one or more treatment plans and in response to the one or more wireless signals received from at least one of: at least one motor module in the at least two motor modules and one or more external devices communicatively coupled to at least one motor module in the at least two motor modules.

16. The method of claim 15, wherein each motor module in the at least two motor modules includes one or more wireless communication components configured to wirelessly communicate with at least one of: at least another motor module in the at least two motor modules and the one or more external devices.

17. (canceled)

18. The method of claim 16, wherein the one or more wireless communication components are configured to wirelessly communicate with at least another wireless communication component in the one or more wireless communication components using one or more communication protocols, the one or more communication protocols include at least one of the following: Bluetooth™ communication protocol, Bluetooth™ Low Energy (BLE) communication protocol, near field communication (NFC) protocol, and any combination thereof.

19. (canceled)

20. The method of claim 15, wherein at least one motor module in the at least two motor modules is selected as a primary motor module and at least another motor module in the at least two motor modules is selected as a secondary motor module, the primary motor module is configured to transmit one or more instructions to the secondary motor module for execution of the treatment plan.

21. (canceled)

22. The method of claim 20, wherein the one or more external devices are wirelessly coupled to the primary motor module, wherein the primary motor module is configured to receive one or more instructions from the one or more external devices and transmit the one or more received instructions to the secondary motor module.

23. The method of claim 22, wherein the one or more external devices are configured to execute at least one of: monitoring, controlling, and adjusting operation of at least one motor module in the at least two motor modules in response to the one or more wireless signals.

24. The method of claim 23, wherein the one or more motors cause one or more motorized struts to execute the at least one adjustment in accordance with the one or more treatment plans, one or more parameters associated with the at least one adjustment include at least one of the following: a direction of the at least one adjustment of the one or more motorized struts, a length of the at least one adjustment of the one or more motorized struts, an angle of the at least one adjustment of the one or more motorized struts, a time for the at least one adjustment of the one or more motorized struts, one or more parameters for changing positioning of the one or more motorized struts, and any combination thereof.

25. (canceled)

26. The method of claim 15, further comprising:
receiving one or more firmware updates using the one or more wireless signals;
authenticating a source of the one or more firmware updates;
verifying the one or more firmware updates;
installing, upon authentication and verification of the one or more firmware updates, the one or more firmware updates on an operating computing system of at least one motor module in the at least two motor modules; and
performing at least one of: the authenticating of the source of the one or more firmware updates, the verifying of the one or more firmware updates, and the installing of the one or more firmware updates without interruption of execution of the at least one adjustment of the one or more motorized struts based on the one or more treatment plans.

27. (canceled)

28. (canceled)
